(19) United States
(12) Patent Application Publication
(10) Pub. No.: US 2004/0077729 A1
(43) Pub. Date: Apr. 22, 2004

(54) New carboxylic acid amides, the preparation thereof and their use as pharmaceutical compositions

(75) Inventors: Uwe Ries, Biberach (DE); Henning Priepke, Warthausen (DE); Wolfgang Wienen, Bieberach/Rissegg (DE); Herbert Nar, Ochsenhausen (DE); Sandra Handschuh, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/624,144

(22) Filed: Jul. 21, 2003

Related U.S. Application Data
(60) Provisional application No. 60/404,430, filed on Aug. 19, 2002.

(30) Foreign Application Priority Data
Jul. 26, 2002 (DE) 102 34 057

(51) Int. Cl. A61K 31/165

(52) U.S. Cl. 514/616; 514/621; 564/155; 564/163; 564/169

(57) ABSTRACT

Carboxylic acid amides of general formula

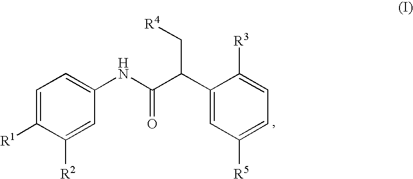

possessing antithrombotic activity and a factor Xa-inhibiting activity. Exemplary are:

(a) 2-(5-amidino-2-hydroxy-phenyl)-N-[3-trifluoromethyl-4-(pyrrolidin-1-yl-carbonyl)phenyl]-3-phenyl-propionamide,

(b) 2-(5-amidino-2-hydroxy-phenyl)-N-[3-trifluoromethyl-4-(pyrrolidin-1-yl-carbonyl)-phenyl]-3-(pyridin-3-yl)-propionamide, and

(c) 2-(5-aminomethyl-2-hydroxy-phenyl)-N-[3-trifluoromethyl-4-(pyrrolidin-l-yl-carbonyl)-phenyl]-3-phenyl-propionamide,

### RELATED APPLICATIONS

Benefit of U.S. Provisional Application Serial No. 60/404,430, filed on Aug. 19, 2002 is hereby claimed.

### FIELD OF THE INVENTION

The present invention relates to new carboxylic acid amides of general formula

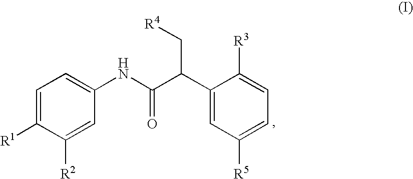

the tautomers, the enantiomers, the diastereomers, the mixtures and the salts thereof, particularly the physiologically acceptable salts thereof with inorganic or organic acids or bases, which have valuable properties.

The compounds of the above general formula I and the tautomers, the enantiomers, the diastereomers, the mixtures and the salts thereof, particularly the physiologically acceptable salts thereof with inorganic or organic acids or bases and the stereoisomers thereof, have valuable pharmacological properties, particularly an antithrombotic effect and a factor Xa-inhibiting effect.

Thus, the present application relates to the new compounds of the above general formula I, the preparation thereof, the pharmaceutical compositions containing the pharmacologically effective compounds, the preparation and use thereof.

In the above general formula

R¹ denotes a C₃₋₇-cycloalkyl-carbonyl group, while

the methylene group in the 3 or 4 position in a C₅₋₇-cycloalkyl-carbonyl group may be replaced by a —NH group, wherein

the hydrogen atom of the —NH group may be replaced by a C₁₋₃-alkyl or C₁₋₃-alkylcarbonyl group,

a C₁₋₆-alkylcarbonyl group, optionally terminally substituted in the alkyl moiety by an amino, C₁₋₃-alkylamino or di-(C₁₋₃-alkyl)-amino group, a group of formula R_{f}R_{g}N—(CH₂)m—(Rₕ)N—CO, wherein

R_{f}, R_{g} and Rₕ independently of one another each denote a hydrogen atom or a C₁₋₃-alkyl group and

m denotes one of the numbers 2, 3 or 4,

a phenylcarbonyl, naphthylcarbonyl or heteroarylcarbonyl group,

while the phenyl, naphthyl or heteroaryl moiety may be substituted by a fluorine, chlorine or bromine atom, by a trifluoromethyl, C₁₋₃-alkyl, amino-C₁₋₃-alkyl, C₁₋₃-alkyl-amino-C₁₋₃-alkyl, di-(C₁₋₃-alkyl)-amino-C₁₋₃-alkyl or C₁₋₃-alkoxy group,

a C₁₋₃-alkyl group substituted by a phenyl or heteroaryl group,

while the phenyl or heteroaryl substituent may be substituted by a fluorine, chlorine or bromine atom, by a trifluoromethyl, C₁₋₃-alkyl, amino-C₁₋₃-alkyl, C₁₋₃-alkylamino-C₁₋₃-alkyl, di-(C₁₋₃-alkyl)-amino-C₁₋₃-alkyl or C₁₋₃-alkoxy group,

a 2,5-dihydro-1H-pyrrol-1-ylcarbonyl group,

a 4- to 7-membered cycloalkyleneimino-carbonyl or cycloalkyleneimino-sulphonyl group optionally substituted by an amino-C₁₋₃-alkyl, C₁₋₃-alkylamino-C₁₋₃-alkyl, di-(C₁₋₃-alkyl)amino-C₁₋₃-alkyl, aminocarbonyl, C₁₋₃-alkylamino-carbonyl or di-(C₁₋₃-alkyl)-aminocarbonyl group or

a group of formula

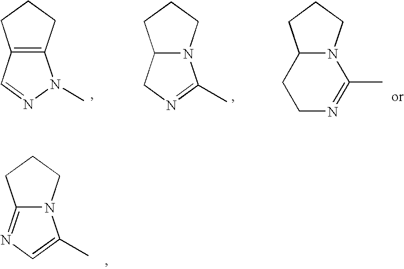

wherein in the heterocyclic moiety a hydrogen atom may be replaced by an aminomethyl or aminocarbonyl group in each case,

R² denotes a fluorine, chlorine or bromine atom, a C₂₋₃-alkenyl group or

a C₁₋₃-alkoxy or C₁₋₃-alkyl group wherein the hydrogen atoms may be wholly or partially replaced by fluorine atoms,

R³ denotes a hydroxy or amino group,

R⁴ denotes a phenyl or heteroaryl group which is optionally substituted by a hydroxy, C₁₋₄-alkyloxy, benzyloxy, hydroxycarbonyl-C₁₋₃-alkoxy, C₁₋₃-alkyloxy-carbonyl-C₁₋₃-alkyloxy, aminocarbonyl-C₁₋₃-alkyloxy, C₁₋₃-alkylaminocarbonyl-C₁₋₃-alkyloxy, di-(C₁₋₃-alkyl)-aminocarbonyl-C₁₋₃-alkyloxy, carboxy, C₁₋₃-alkyloxy-carbonyl group,

a 1-H-pyridonyl or 1-(C₁₋₃-alkyl)-pyridonyl group,

a 4- to 7-membered cycloalkyleneimino group or

a 4- to 7-membered cycloalkyl group wherein one or two methylene groups are replaced by a —NH or —N(C₁₋₃-alkyl)-group and wherein one or two of the methylene groups adjacent to the —NH or —N(C₁₋₃-alkyl)-group may each be replaced by a carbonyl group, with the proviso that a cycloalkyl group as hereinbefore defined wherein two —NH or —N(C₁₋₃-alkyl)-groups are separated from one another by precisely one —CH₂— group is excluded, and

R⁵ denotes a group of formula —CH₂—NHR⁶, wherein

R⁶ denotes a hydrogen atom, a C₁₋₁₀-alkoxy-carbonyl, 2,2,2-trichloroethoxy-carbonyl, phenyloxycarbonyl or benzyloxycarbonyl group,

or a group of formula —C(═NH)—NH₂ wherein a hydrogen atom may be replaced by a C₁₋₁₀-alkoxy-carbonyl, 2,2,2-trichloroethoxycarbonyl, phenyloxycarbonyl, benzyloxycarbonyl, phenylcarbonyl, hydroxy, C₁₋₅-alkyloxy, benzyloxy or phenyloxy group,

while, unless otherwise stated, the term heteroaryl group denotes a monocyclic 5- or 6-membered heteroaryl group optionally substituted in the carbon skeleton by a C₁₋₃-alkyl, carboxy, C₁₋₃-alkoxy-carbonyl or C₁₋₃-alkoxy-carbonylamino group, while

the 6-membered heteroaryl group contains one, two or three nitrogen atoms and

the 5-membered heteroaryl group contains an imino group optionally substituted by a C₁₋₃-alkyl or phenyl-C₁₋₃-alkyl group, an oxygen or sulphur atom or

an imino group optionally substituted by a C₁₋₃-alkyl, amino-C₁₋₃-alkyl, C₁₋₃-alkylamino-C₁₋₃-alkyl, di-(C₁₋₃-alkyl)-amino-C₁₋₃-alkyl or phenyl-C₁₋₃-alkyl group or an oxygen or sulphur atom and additionally contains a nitrogen atom or

an imino group optionally substituted by a C₁₋₃-alkyl or phenyl-C₁₋₃-alkyl group or an oxygen or sulphur atom and additionally contains two nitrogen atoms,

an imino group optionally substituted by a C₁₋₃-alkyl or phenyl-C₁₋₃-alkyl group and contains three nitrogen atoms,

and moreover a phenyl ring may be fused to the abovementioned monocyclic heterocyclic groups via two adjacent carbon atoms and the binding takes place via a nitrogen atom or via a carbon atom of the heterocyclic moiety or a fused-on phenyl ring,

while the abovementioned alkyl and alkoxy groups include straight-chain and branched alkyl and alkoxy groups, wherein additionally one to 3 hydrogen atoms may be replaced by fluorine atoms.

Those compounds of general formula (I) wherein R¹ to R⁴ are as hereinbefore defined and R⁵ denotes an aminomethyl group substituted at the nitrogen atom by a C₁₋₁₀-alkoxy-carbonyl, 2,2,2-trichloroethoxycarbonyl, phenyloxycarbonyl or benzyloxycarbonyl group or an amidino group substituted by a C₁₋₁₀-alkoxy-carbonyl, 2,2,2-trichloroethoxycarbonyl, phenyloxycarbonyl, benzyloxycarbonyl, phenylcarbonyl, hydroxy, C₁₋₅-alkyloxy, benzyloxy or phenyloxy group, are prodrugs for those compounds of general formula (I) wherein R⁵ denotes an aminomethyl or amidino group.

Preferred compounds of the above general formula I are those wherein

R², R³, R⁴ and R⁵ are as hereinbefore defined and

R¹ denotes a 2,5-dihydro-1H-pyrrol-1-ylcarbonyl group, a 4- to 7-membered cycloalkyleneimino-carbonyl group optionally substituted by an amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group or a group of formula

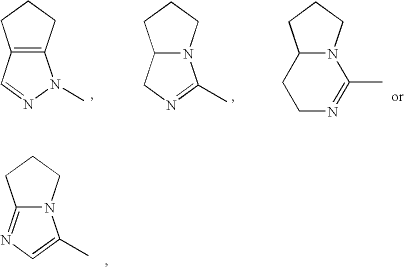

wherein in the heterocyclic moiety a hydrogen atom may be replaced by an aminomethyl or aminocarbonyl group in each case,

the abovementioned alkyl and alkoxy groups including straight-chain and branched alkyl and alkoxy groups, wherein additionally one to 3 hydrogen atoms may be replaced by fluorine atoms,

the tautomers, enantiomers, the diastereomers, the mixtures and the salts thereof.

Particularly preferred are those compounds of the above general formula I, wherein

R¹, R², R³ and R⁵ are as hereinbefore defined and

R⁴ denotes a phenyl, furanyl, thienyl, pyrrolyl, pyrazolyl, pyridinyl, pyrazinyl, pyridazinyl, pyrimidinyl, thiazolyl or isoxazolyl group which is optionally substituted by a hydroxy, C₁₋₄-alkyloxy, benzyloxy, hydroxycarbonyl-C₁₋₃-alkoxy, C₁₋₃-alkyloxycarbonyl-C₁₋₃-alkyloxy, aminocarbonyl-C₁₋₃-alkyloxy, C₁₋₃-alkylaminocarbonyl-C₁₋₃-alkyloxy, di-(C₁₋₃-alkyl)-aminocarbonyl-C₁₋₃-alkyloxy, carboxy, C₁₋₃-alkyloxy-carbonyl group,

the abovementioned alkyl and alkoxy groups including straight-chain and branched alkyl and alkoxy groups, wherein additionally one to 3 hydrogen atoms may be replaced by fluorine atoms,

the tautomers, enantiomers, the diastereomers, the mixtures and the salts thereof.

The following preferred compounds of general formula I are mentioned by way of example:

(1) 2-(5-amidino-2-hydroxy-phenyl)-N-[3-trifluoromethyl-4-(pyrrolidin-1-yl-carbonyl)-phenyl]-3-phenyl-propionamide,

(2) 2-(5-amidino-2-hydroxy-phenyl)-N-[3-trifluoromethyl-4-(pyrrolidin-1-yl-carbonyl)-phenyl]-3-(pyridin-3-yl)-propionamide and

(3) 2-(5-aminomethyl-2-hydroxy-phenyl)-N-[3-trifluoromethyl-4-(pyrrolidin-1-ylcarbonyl)-phenyl]-3-phenyl-propionamide,

wherein the amidino group may additionally be substituted by a hydroxy, C₁₋₅-alkyloxy, C₁₋₁₀-alkoxy-carbonyl or phenylcarbonyl group, and the salts thereof.

According to the invention the compounds of general formula I are obtained by methods known per se, for example by the following methods:

a) acylation of a compound of general formula

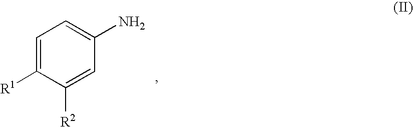

wherein R¹ and R² are as hereinbefore defined, with a carboxylic acid or with a reactive carboxylic acid derivative of general formula

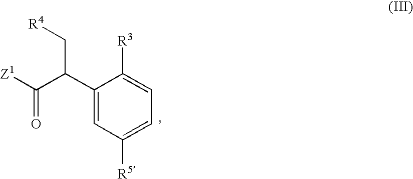

wherein R³ and R⁴ are as hereinbefore defined, the groups R³ and R⁴ optionally being protected before the reaction by suitable protective groups which are cleaved again after the reaction,

R^{5′} denotes a cyano group, a protected aminomethyl group or an amidino group protected by a carbamate group and

Z¹ denotes a hydroxy group or a nucleofugic leaving group such as for example a C₁₋₆-alkoxy-carbonyloxy, C₁₋₆-alkyl-carbonyloxy or 2,6-dichlorophenylcarbonyloxy group or a chlorine or bromine atom, and subsequently converting the resulting cyano compound into an amidino or aminomethyl compound optionally substituted with the groups mentioned hereinbefore.

The acylation is conveniently carried out with a corresponding halide or anhydride in a solvent such as methylene chloride, chloroform, carbon tetrachloride, ether, tetrahydrofuran, dioxane, benzene, toluene, acetonitrile, dimethylformamide or sulpholane, optionally in the presence of an inorganic or organic base at temperatures between −20 and 200° C., but preferably at temperatures between −10 and 160° C.

The acylation may however also be carried out with the free acid or an ester, optionally in the presence of an acid-activating agent or a dehydrating agent, e.g. in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, triethylamine, N,N′-dicyclohexylcarbodiimide, N,N′-dicyclohexylcarbodiimide/N-hydroxysuccinimide or 1-hydroxy-benzotriazole, O-(benzotriazol-1-yl)-N,N,N′,N′-tetramethyluronium tetrafluoroborate/N-methylmorpholine, propanephosphonic acid-cyclo-anhydride/N-methylmorpholine, N,N′-carbonyldiimidazole or N,N′-thionyldiimidazole or triphenylphosphine/carbon tetrachloride, at temperatures between −20 and 200° C., but preferably at temperatures between −10 and 160° C.

Other methods of amide coupling are described for example in P. D. Bailey, I. D. Collier, K. M. Morgan in Comprehensive Functional Group Interconversions Vol.5, 257ff. Pergamon, 1995.

The subsequent conversion of the cyano group into an amidino group is carried out as described in process b).

b) In order to prepare a compound of general formula I wherein R₅ denotes an amidino group optionally substituted by a C₁₋₁₀-alkoxy-carbonyl, 2,2,2-trichloroethoxycarbonyl, phenyloxycarbonyl, benzyloxycarbonyl, phenylcarbonyl, hydroxy, C₁₋₅-alkyloxy, benzyloxy or phenyloxy group:

Reacting a compound of general formula

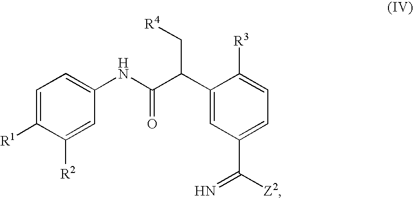

optionally formed in the reaction mixture,

wherein

R¹ to R⁴ are as hereinbefore defined and

Z² denotes an alkoxy, aralkoxy, alkylthio or aralkylthio group, with an amine of general formula

H—R⁷NR⁸, (V)

wherein

R⁷ denotes a hydrogen atom and

R⁸ denotes a hydrogen atom or a hydroxy, C₁₋₅-alkyloxy, benzyloxy or phenyloxy group, or with the salts thereof.

The reaction is expediently carried out in a solvent such as methanol, ethanol, n-propanol, tetrahydrofuran or dioxane at temperatures between 0 and 150° C., preferably at temperatures between 0 and 80° C., with an amine of general formula V or with a corresponding acid addition salt such as for example ammonium carbonate or ammonium acetate.

A compound of general formula IV is obtained for example by reacting a corresponding cyano compound with a corresponding alcohol such as methanol, ethanol, n-propanol, isopropanol or benzylalcohol in the presence of an acid such as hydrochloric acid or by reacting a corresponding amide with a trialkyloxonium salt such as triethyloxonium-tetrafluoroborate in a solvent such as methylene chloride, tetrahydrofuran or dioxane at temperatures between 0 and 50° C., but preferably at 20° C., or a corresponding nitrile with hydrogen sulphide, conveniently in a solvent such as pyridine or dimethylformamide and in the presence of a base such as triethylamine, and subsequently alkylating the thioamide formed with a corresponding alkyl or aralkyl halide.

c) In order to prepare a compound of general formula I wherein R₅ denotes an aminomethyl group optionally substituted at the nitrogen atom by a C₁₋₁₀-alkoxy-carbonyl, 2,2,2-trichloroethoxycarbonyl, phenyloxycarbonyl or benzyloxycarbonyl group:

Catalytically hydrogenating a compound of general formula

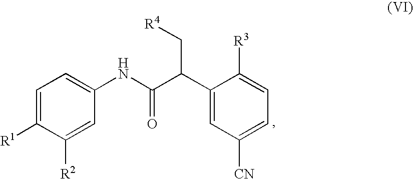

wherein

R¹ to R⁴ are as hereinbefore defined,

and optionally subsequently reacting the aminomethyl compound thus obtained with a compound of formula

R⁹—Z³ (VII),

wherein R⁹ denotes a C₁₋₁₀-alkoxy-carbonyl, 2,2,2-trichloroethoxycarbonyl, phenyloxycarbonyl or benzyloxycarbonyl group and Z³ denotes a leaving group such as a halogen atom or a sulphonyloxy group, e.g. a chlorine, bromine or iodine atom or a trifluoromethylsulphonyloxy group.

The catalytic hydrogenation is carried out with hydrogen in the presence of a catalyst such as palladium/charcoal, platinum in a solvent such as methanol, ethanol, ethyl acetate, dimethylformamide, dimethylformamide/acetone or glacial acetic acid, optionally with the addition of an acid such as hydrochloric acid, at temperatures between 0 and 50° C., but preferably at ambient temperature, and under a hydrogen pressure of 1 to 7 bar, but preferably from 3 to 5 bar, or for example with Raney nickel, preferably in methanolic ammonia solution.

The optional subsequent acylation may be carried out as described in process a).

If according to the invention a compound of general formula I is obtained which contains an amino or imino group, this may then be converted with a corresponding acyl-derivative into a corresponding acyl compound of general formula I and/or

if a compound of general formula I is obtained which contains an esterified carboxy group, this may then be converted by hydrolysis into a corresponding carboxylic acid of general formula I and/or

if a compound of general formula I is obtained which contains a carboxy group, this may then be converted by esterification into a corresponding ester.

The subsequent acylation is conveniently carried out with a corresponding halide or anhydride in a solvent such as methylene chloride, chloroform, carbon tetrachloride, ether, tetrahydrofuran, dioxane, benzene, toluene, acetonitrile or sulpholane, optionally in the presence of an inorganic or organic base at temperatures between −20 and 200° C., but preferably at temperatures between −10 and 160° C. However, it may also be carried out with the free acid, optionally in the presence of an acid-activating agent or a dehydrating agent, e.g. in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/-N-hydroxysuccinimide or 1-hydroxy-benzotriazole, N,N′-carbonyldiimidazole or N,N′-thionyldiimidazole or triphenylphosphine/carbon tetrachloride, at temperatures between −20 and 200° C., but preferably at temperatures between −10 and 160° C. As a result of the acylation the C₁₋₁₀-alkoxy-carbonyl, 2,2,2-trichloroethoxycarbonyl, phenyloxycarbonyl, benzyloxycarbonyl or phenylcarbonyl group may be introduced, for example.

The subsequent hydrolysis is conveniently carried out either in the presence of an acid such as hydrochloric acid, sulphuric acid, phosphoric acid, acetic acid, trichloroacetic acid, trifluoroacetic acid or mixtures thereof or in the presence of a base such as lithium hydroxide, sodium hydroxide or potassium hydroxide in a suitable solvent such as water, water/methanol, water/ethanol, water/isopropanol, methanol, ethanol, water/tetrahydrofuran or water/dioxane and the subsequent decarboxylation is carried out in the presence of an acid as hereinbefore described at temperatures between −10 and 120° C., e.g. at temperatures between ambient temperature and the boiling temperature of the reaction mixture.

The subsequent esterification is carried out with a corresponding alcohol, conveniently in a solvent or mixture of solvents such as methylene chloride, benzene, toluene, chlorobenzene, tetrahydrofuran, benzene/tetrahydrofuran or dioxane, but preferably in an excess of the alcohol used, optionally in the presence of an acid such as hydrochloric acid or in the presence of a dehydrating agent, e.g. in the presence of isobutyl chloroformate, thionyl chloride, trimethylchlorosilane, hydrochloric acid, sulphuric acid, methanesulphonic acid, p-toluenesulphonic acid, phosphorus trichloride, phosphorus pentoxide, N,N′-dicyclohexylcarbodiimide, N,N′-dicyclohexylcarbodiimide/N-hydroxysuccinimide, N,N′-carbonyldiimidazole or N,N′-thionyldiimidazole, triphenylphosphine/carbon tetrachloride or triphenylphosphine/diethyl azodicarboxylate, optionally in the presence of a base such as potassium carbonate, N-ethyl-diisopropylamine or N,N-dimethylamino-pyridine, conveniently at temperatures between 0 and 150° C., preferably at temperatures between 0 and 80° C., or with a corresponding halide in a solvent such as methylene chloride, tetrahydrofuran, dioxane, dimethylsulphoxide, dimethylformamide or acetone, optionally in the presence of a reaction accelerator such as sodium or potassium iodide and preferably in the presence of a base such as sodium carbonate or potassium carbonate or in the presence of a tertiary organic base such as N-ethyl-diisopropylamine or N-methyl-morpholine, which may simultaneously also serve as solvent, or optionally in the presence of silver carbonate or silver oxide at temperatures between −30 and 100° C., but preferably at temperatures between −10 and 80° C.

In the reactions described above any reactive group present such as hydroxy, carboxy, amino, alkylamino or imino groups may be protected during the reaction by conventional protecting groups which are cleaved again after the reaction.

For example, a suitable protecting group for a hydroxy group may be the methoxy, benzyloxy, trimethylsilyl, acetyl, benzoyl, tert.butyl, trityl, benzyl or tetrahydropyranyl group,

suitable protecting groups for a carboxyl group might be the trimethylsilyl, methyl, ethyl, tert.butyl, benzyl or tetrahydropyranyl group, and

suitable protecting groups for an amino, alkylamino or imino group might be the acetyl, trifluoroacetyl, benzoyl, ethoxycarbonyl, tert.butoxycarbonyl, benzyloxycarbonyl, benzyl, methoxybenzyl or 2,4-dimethoxybenzyl group and additionally, for the amino group, the phthalyl group.

Other protecting groups and their cleaving are described in T. W. Greene, P. G. M. Wuts, Protective Groups in Synthesis, Wiley, 1991.

Any protecting group used may optionally subsequently be cleaved for example by hydrolysis in an aqueous solvent, e.g. in water, isopropanol/water, tetrahydrofuran/water or dioxane/water, in the presence of an acid such as trifluoroacetic acid, hydrochloric acid or sulphuric acid or in the presence of an alkali metal base such as lithium hydroxide, sodium hydroxide or potassium hydroxide or by ether splitting, e.g. in the presence of iodotrimethylsilane, at temperatures between 0 and 100° C., preferably at temperatures between 10 and 50° C.

However, a benzyl, methoxybenzyl or benzyloxycarbonyl group is preferably cleaved hydrogenolytically, e.g. with hydrogen in the presence of a catalyst such as palladium/charcoal in a solvent such as methanol, ethanol, ethyl acetate, dimethylformamide, dimethylformamide/acetone or glacial acetic acid, optionally with the addition of an acid such as hydrochloric acid at temperatures between 0 and 50° C., but preferably at ambient temperature, and at a hydrogen pressure of 1 to 7 bar, but preferably 3 to 5 bar.

A methoxybenzyl group may also be cleaved in the presence of an oxidising agent such as cerium(IV)ammonium nitrate in a solvent such as methylene chloride, acetonitrile or acetonitrile/water at temperatures between 0 and 50° C., but preferably at ambient temperature.

A methoxy group is conveniently cleaved in the presence of boron tribromide in a solvent such as methylene chloride at temperatures between −35 and −25° C.

However, a 2,4-dimethoxybenzyl group is preferably cleaved in trifluoroacetic acid in the presence of anisole.

A tert.butyl or tert.butyloxycarbonyl group is preferably cleaved by treating with an acid such as trifluoroacetic acid or hydrochloric acid, optionally using a solvent such as methylene chloride, dioxane or ether.

A phthalyl group is preferably cleaved in the presence of hydrazine or a primary amine such as methylamine, ethylamine or n-butylamine in a solvent such as methanol, ethanol, isopropanol, toluene/water or dioxane at temperatures between 20 and 50° C.

An allyloxycarbonyl group is cleaved by treating with a catalytic amount of tetrakis(triphenylphosphine)-palladium(0), preferably in a solvent such as tetrahydrofuran and preferably in the presence of an excess of a base such as morpholine or 1,3-dimedone at temperatures between 0 and 100° C., preferably at ambient temperature and under an inert gas, or by treating with a catalytic amount of tris-(triphenylphosphine)-rhodium(I)-chloride in a solvent such as aqueous ethanol and optionally in the presence of a base such as 1,4-diazabicyclo[2.2.2]octane at temperatures between 20 and 70° C.

The compounds of general formulae II to VI used as starting materials which are known from the literature are obtained by methods known from the literature and also their preparation is described in the Examples.

For example, a compound of general formula III may be prepared by deprotonating a compound of general formula

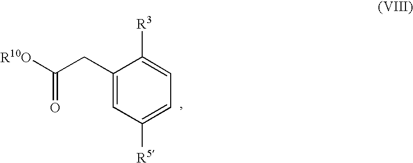

wherein

R³ is as hereinbefore defined, R³ optionally being protected prior to the reaction by a suitable protecting group which is cleaved again after the reaction,

R^{5′} denotes a cyano group and

R¹⁰OCO denotes an optionally protected carboxy group, while R¹⁰ denotes hydrogen or a suitable protecting group such as for example a C₁₋₆-alkyl group, in the benzyl position and then alkylating by reacting with a compound of general formula

R⁴—CH₂—Z⁴ (IX),

wherein R⁴ is as hereinbefore defined and Z⁴ denotes a nucleofugic leaving group such as for example a chlorine, bromine or iodine atom or a p-tolylsulphonyl, methylsulphonyl or trifluoromethylsulphonyl group. The deprotonation is carried out using suitable bases such as for example sodium hydride, potassium tert.butoxide or lithium diisopropylamine in solvents such as for example DMF, tetrahydrofuran, dimethylsulphoxide, ether or mixtures thereof at temperatures between −10 and 20° C., preferably 5 to 15° C.

The preparation of carboxylic acid derivatives of general formula III is described in Methoden der organischen Chemie (Houben-Weyl), Volume E5, “Carboxylic acids and carboxylic acid derivatives”, 4th edition, Verlag Thieme, Stuttgart 1985.

Moreover, the compounds of general formula I obtained may be resolved into their enantiomers and/or diastereomers.

Thus, for example, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical enantiomers and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be, for example, (+) or (−)-menthol and an optically active acyl group in amides, for example, may be a (+)- or (−)-menthyloxycarbonyl.

Furthermore, the compounds of formula I may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, if the new compounds of formula I thus obtained contain a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine and triethanolamine.

As already mentioned, the new compounds of general formula I and the tautomers, the enantiomers, the diastereomers and the physiologically acceptable salts thereof have valuable pharmacological properties, particularly an antithrombotic effect which is preferably based on a factor Xa-influencing effect, for example a factor Xa-inhibiting effect, and on an inhibiting effect on related serine proteases such as, for example, thrombin, trypsin, urokinase, factor VIIa, factor IXa, factor XIa and factor XIIa.

For example the compound

(A) 2-(5-amidino-2-hydroxy-phenyl)-N-[3-trifluoromethyl-4-(pyrrolidin-1-yl-carbonyl)-phenyl]-3-(pyridin-3-yl)-propionamide-dihydrochloride was investigated for its effect on the inhibition of factor Xa as follows:

Method: Enzyme-kinetic measurement with chromogenic substrate. The quantity of p-nitroaniline (pNA) released from the colourless chromogenic substrate by human factor Xa is determined photometrically at 405 nm. It is proportional to the activity of the enzyme used. The inhibition of the enzyme activity by the test substance (in relation to the solvent control) is determined at various concentrations of test substance and from this the IC₅₀ is calculated, as the concentration which inhibits the factor Xa used by 50%.

Material:

Tris(hydroxymethyl)-aminomethane buffer (100 mmol) and sodium chloride (150 mmol), pH 8.0

Factor Xa (Roche), spec. activity: 10 U/0.5 ml, final concentration: 0.175 U/ml for each reaction mixture

Substrate Chromozym X (Roche), final concentration: 200 μMol/l for each reaction mixture

Test substance: final concentration 100, 30, 10, 3, 1, 0.3, 0.1, 0.03, 0.01, 0.003, 0.001 μMol/l

Procedure:

10 μl of a 23.5-times concentrated starting solution of the test substance or solvent (control), 175 μl of tris(hydroxymethyl)-aminomethane buffer and 25 μl of a 1.65 U/ml Factor Xa working solution are incubated for 10 minutes at 37° C. After the addition of 25 μl of Chromozym X working solution (1.88 μmol/l) the sample is measured in a photometer (SpectraMax 250) at 405 nm for 150 seconds at 37° C.

Evaluation:

1. Determining the maximum increase (deltaOD/minutes) over 3 measuring points.

2. Determining the % inhibition based on the solvent control.

3. Plotting a dosage/activity curve (% inhibition vs substance concentration).

4. Determining the IC₅₀ by interpolating the X-value (substance concentration) of the dosage/activity curve at Y=50% inhibition.

The Table that follows contains the results found:

| | inhibition of factor Xa |
|---|---|
| substance | (IC₅₀ in μM) |
| (A) | 0.007 |

The compounds prepared according to the invention are generally well tolerated.

In view of their pharmacological properties the new compounds and the physiologically acceptable salts thereof are suitable for the prevention and treatment of venous and arterial thrombotic diseases, such as for example the prevention and treatment of deep leg vein thrombosis, for preventing reocclusions after bypass operations or angioplasty (PT(C)A), and occlusion in peripheral arterial diseases, and for the prevention and treatment of pulmonary embolism, disseminated intravascular coagulation, for preventing and treating coronary thrombosis, for preventing stroke and the occlusion of shunts. In addition, the compounds according to the invention are suitable for antithrombotic back-up in thrombolytic treatment, such as for example with alteplase, reteplase, tenecteplase, staphylokinase or streptokinase, for preventing long-term restenosis after PT(C)A, for the prevention and treatment of ischaemic incidents in patients with all forms of coronary heart disease, for preventing metastasis and the growth of tumours and inflammatory processes, e.g. in the treatment of pulmonary fibrosis, for preventing and treating rheumatoid arthritis, for preventing and treating fibrin-dependent tissue adhesions and/or the formation of scar tissue and for promoting wound healing processes. The new compounds and the physiologically acceptable salts thereof may be used therapeutically in conjunction with acetylsalicylic acid, with inhibitors of platelet aggregation such as fibrinogen receptor antagonists (e.g. abciximab, eptifibatide, tirofiban, roxifiban), with physiological activators and inhibitors of the clotting system and the recombinant analogues thereof (e.g. Protein C, TFPI, antithrombin), with inhibitors of ADP-induced aggregation (e.g. clopidogrel, ticlopidine), with P₂T receptor antagonists (e.g. cangrelor) or with combined thromboxane receptor antagonists/synthetase inhibitors (e.g. terbogrel).

The dosage required to achieve such an effect is appropriately 0.01 to 3 mg/kg, preferably 0.03 to 1.0 mg/kg by intravenous route, and 0.03 to 30 mg/kg, preferably 0.1 to 10 mg/kg by oral route, in each case administered 1 to 4 times a day. For this purpose, the compounds of formula I prepared according to the invention may be formulated, optionally together with other active substances, with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The Examples which follow are intended to illustrate the invention without restricting its scope:

### EXAMPLE 1

### 2-(5-Amidino-2-hydroxy-phenyl)-N-[3-trifluoromethyl-4-(pyrrolidin-1-yl-carbonyl)phenyl]-3-phenyl-propionamide-hydrochloride

a. 4-nitro-2-trifluoromethyl-benzonitrile

30.9 g (0.15 mol) of 2-amino-5-nitro-benzotrifluoride are suspended in 335 ml of water and 66.5 ml of conc. sulphuric acid. At 0° C. a solution of 10.7 g (0.156 mol) of sodium nitrite in 40 ml of water is added dropwise and once it has all been added the mixture is stirred for another 20 minutes. The undissolved material is suction filtered, the diazonium salt solution is obtained.

45 g (0.18 mol) of copper sulphate×5 H₂O are dissolved in 500 ml of water, then 52.5 g (0.8 mol) of potassium cyanide are added while gently cooling with ice and stirred for 20 minutes. Then a solution of 225 g (2.1 mol) of sodium carbonate in 450 ml of water is added, and at 0° C. the diazonium salt solution is added dropwise. After the reaction has warmed up to ambient temperature, stirring is continued for another 2 hours at 45° C. and then the mixture is extracted with ethyl acetate. The combined organic extracts are dried over sodium sulphate and concentrated by evaporation. The crude product is chromatographed on silica gel, eluting with petroleum ether/ethyl acetate (9/1 to 8/2).

Yield: 6.9 g (18% of theory),

R_{f} value: 0.5 (silica gel; petroleum ether/ethyl acetate=8:2).

b. 4-nitro-2-trifluoromethyl-benzoic acid

2.1 g (10 mmol) of 4-nitro-2-trifluoromethyl-benzonitrile are dissolved in 20 ml of 55% sulphuric acid and stirred for 2 hours at 165° C. Then the mixture is cooled, adjusted with sodium hydroxide solution to pH 8-9 and extracted with ethyl acetate. The combined organic extracts are dried and concentrated by evaporation. The residue is chromatographed on silica gel, eluting with dichloromethane/methanol 50:1.

Yield: 1.0 g (45% of theory),

R_{f} value: 0.1 (silica gel; dichloromethane/ethanol=9:1).

c. 3-trifluoromethyl-4-(pyrrolidin-1-yl-carbonyl)-nitrobenzene

3.9 g (16.5 mmol) of 4-nitro-2-trifluoromethyl-benzoic acid are dissolved in 75 ml of dimethylformamide and after the addition of 5.9 g (18 mmol) of O-(benzotriazol-1-yl)N,N,N′,N′-tetramethyluroniumtetrafluoroborate, 2.0 ml (18.5 mmol) of N-methylmorpholine and 1.5 ml (18 mmol) of pyrrolidine the mixture is stirred for 16 hours at ambient temperature. The reaction product is stirred into ice water/1 M hydrochloric acid and extracted with ethyl acetate. The combined organic extracts are dried and concentrated by evaporation. The residue is crystallised from ether/petroleum ether.

Yield: 4.1 g (80% of theory),

R_{f} value: 0.6 (silica gel; dichloromethane/ethanol=9:1).

d. 3-trifluoromethyl-4-(pyrrolidin-1-yl-carbonyl)-aniline

2.6 g (7.4 mmol) of 3-trifluoromethyl-4-(pyrrolidin-1-yl-carbonyl)-nitrobenzene are dissolved in 50 ml ethyl acetate and 50 ml of methanol and after the addition of 1 g palladium on activated charcoal (10%) hydrogenated with hydrogen at ambient temperature. Then the catalyst is filtered off and the mixture is concentrated by evaporation. The residue is recrystallised from ethyl acetate.

Yield: 1.6 g (87% of theory),

R_{f} value: 0.45 (silica gel; dichloromethane/ethanol=9:1).

e. methyl 2-(2-benzyloxy-5-cyano-phenyl)-3-phenyl-propionate

1.0 g (3.5 mmol) of methyl (2-benzyloxy-5-cyano-phenyl)-acetate are suspended in 25 ml of tetrahydrofuran and at 10° C. 120 mg (4.7 mmol) of sodium hydride are added batchwise. After 45 minutes 0.5 ml (4.6 mmol) of benzylbromide are added dropwise and the mixture is stirred for 16 hours at ambient temperature. The tetrahydrofuran is distilled off, the residue is distributed in ethyl acetate / water, the organic phase is washed with sodium hydrogen carbonate solution, dried and concentrated by evaporation.

Yield: 1.5 g (97% of theory),

R_{f} value: 0.52 (silica gel; petroleum ether/ethyl acetate=7:3).

f. 2-(2-benzyloxy-5-cyano-phenyl)-3-phenyl-propionic acid

1.5 g (4 mmol) of methyl 2-(2-benzyloxy-5-cyano-phenyl)-3-phenyl-propionate are dissolved in 25 ml of methanol and after the addition of 10 ml of 1 M sodium hydroxide solution the mixture is stirred for 3 h at 60° C. The methanol is distilled off, the residue is taken up in water and extracted with ether. The aqueous phase is adjusted to pH 6 with glacial acetic acid and the product precipitated is suction filtered.

Yield: 1.0 g (69% of theory),

melting point: 170-171 ° C.

g. 2-(2-benzyloxy-5-cyano-phenyl)-3-phenyl-propionic acid chloride

1.00 g (2.8 mmol) of 2-(2-benzyloxy-5-cyano-phenyl)-3-phenyl-propionic acid are dissolved in 10 ml of dichloromethane and after the addition of 0.6 ml (8.4 mmol) of thionyl chloride and 0.1 ml of dimethylformamide the mixture is refluxed for 1. 5 hours. Then the reaction mixture is concentrated by evaporation and further reacted without any purification.

Yield: 1.05 g ( 100% of theory).

h. 2-(2-benzyloxy-5-cyano-phenyl)-N-[3-trifluoromethyl-4-pyrrolidin-1-yl-carbonyl)-phenyl]-3-phenyl-propionamide

A solution of 413 mg (1.1 mmol) of 2-(2-benzyloxy-5-cyano-phenyl)-3-phenylpropionic acid chloride in 30 ml of tetrahydrofuran is combined with 258 mg (1 mmol) of 3-trifluoro-methyl-4-(pyrrolidin-1-yl-carbonyl)-aniline. Then 0.3 ml (3 mmol) of triethylamine are added dropwise. After 14 hours at ambient temperature the mixture is poured onto 100 ml of ice water and extracted with ethyl acetate. The organic phase is washed again with hydrochloric acid, dried and concentrated by evaporation. The residue is chromatographed on silica gel, eluting with petroleum ether/ethyl acetate 1:1.

Yield: 0.50 g (84% of theory),

R_{f} value: 0.35 (silica gel; petroleum ether/ethyl acetate=2:1).

i. 2-(2-benzyloxy-5-amidino-phenyl)-N-[3-trifluoromethyl-4-(pyrrolidin-1-yl-carbonyl)-phenyl]-3-phenyl-propionamide-hydrochloride

500 mg (0.8 mmol) of 2-(2-benzyloxy-5-cyano-phenyl)-N-[3-trifluoromethyl-4-pyrrolidin-1-yl-carbonyl)-phenyl]-3-phenyl-propionamide are dissolved in 30 ml of saturated ethanolic hydrochloric acid and stirred for 17 hours at ambient temperature. The solvent is distilled off, the residue is dissolved in 20 ml of absolute ethanol and combined with 1.2 g (12.5 mmol) of ammonium carbonate. After 22 hours the mixture is evaporated to dryness. The residue is combined with ethanol, the insoluble inorganic salts are suction filtered, the filtrate is combined with ether and the precipitated product is suction filtered.

Yield: 0.45 g ( 83% of theory),

R_{f} value: 0.26 (silica gel; petroleum ether/ethyl acetate=1:1) C₃₅H₃₃F₃N_{4O}₃×HCl (614.67/651.134).

| | |
|---|---|
| Mass spectrum: | (M + H)⁺ = 615 |
| | (M + Cl)⁻ = 649/51 (chlorine isotope) |

j. 2-(5-amidino-2-hydroxy-phenyl)-N-[3-trifluoromethyl-4-(pyrrolidin-1-yl-carbonyl)-phenyl]-3-phenyl-propionamide-hydrochloride

Prepared analogously to Example 1d from 2-(2-benzyloxy-5-carbamimidoyl-phenyl)-N[3-trifluoromethyl-4-(pyrrolydin-1-yl-carbonyl)-phenyl]-3-phenyl-propionamide and palladium on activated charcoal in methanol.

Yield: 80% of theory

R_{f} value: 0.66 (Reversed phase RP 8; 5% sodium chloride solution/methanol=1:3) C₂₈H₂₇F₃N₄O₃×HCl (524.54/561.009)

| | |
|---|---|
| Mass spectrum: | (M + H)⁺ = 525 |
| | (M − H)⁻ = 523 |
| | (M + Cl)⁻ = 559/61 (chlorine isotope) |

### EXAMPLE 2

### 2-(5-amidino-2-hydroxy-phenyl)-N-[3-trifluoromethyl-4-(pyrrolidin-1-yl-carbonyl)phenyl]-3-(pyridin-3-yl)-propionamide-dihydrochloride

Prepared analogously to Example 1k from 2-(5-amidino-2-benzyloxy-phenyl)-N-[3-trifluoromethyl-4-(pyrrolidin-1-yl-carbonyl)-phenyl]-3-(pyridin-3-yl)-propionamide-dihydrochloride and palladium on activated charcoal in methanol.

Yield: 66% of theory,

R_{f} value: 0.42 (Reversed phase RP 8; 5% sodium chloride solution/methanol=2:3) C₂₇H₂₉N₅O₃×2 HCl (471.55/544.48)

| | |
|---|---|
| Mass spectrum: | (M + H)⁺ = 472 |

### EXAMPLE 3

### 2-(5-Aminomethyl-2-hydroxy-phenyl)-N-[3-trifluoromethyl-4-(pyrrolidin-1-yl-carbonyl)-phenyl]-3-propionamide

a. 2-(5-aminomethyl-2-benzyloxy-phenyl)-N-[3-trifluoromethyl-4-(pyrrolidin-1-yl-carbonyl)-phenyl]-3-phenyl-propionamide

580 mg (0.97 mmol) of 2-(2-benzyloxy-5-cyano-phenyl)-N-[3-trifluoromethyl-4-pyrrolidin-1-yl-carbonyl)-phenyl]-phenyl-propionamide (Example 1h) are dissolved in 40 ml of methanolic ammonia and after the addition of 400 mg of Raney nickel hydrogenated with hydrogen for 5 hours at ambient temperature. The catalyst is filtered off and the solution is concentrated by evaporation.

Yield: 580 mg (100% of theory),

R_{f} value: 0.2 (silica gel; dichloromethane/methanol=4: 1)

b. 2-(5-aminomethyl-2-hydroxy-phenyl)-N-[3-trifluoromethyl-4-(pyrrolidin-1-yl-carbonyl)-phenyl]-3-phenyl-propionamide

Prepared analogously to Example 1k from 2-(5-aminomethyl-2-benzyloxy-phenyl)-N-[3-trifluoromethyl-4-(pyrrolidin-1-yl-carbonyl)-phenyl]-3-phenyl-propionamide and palladium on activated charcoal in methanol.

Yield: 75% of theory,

R_{f} value: 0.75 (silica gel; dichloromethane/ethanol/ammonia=4:1:0.1); C₂₈H₂₈F₃N₃O₃ (511.549)

| | |
|---|---|
| Mass spectrum: | (M + H)⁺ = 512 |
| | (M − H)⁻ = 510 |

### EXAMPLE 4

Dry Ampoule Containing 75 mg of Active Substance per 10 ml

Composition:

| | | |
|---|---|---|
| Active substance | 75.0 | mg |
| Mannitol | 50.0 | mg |
| water for injections | ad 10.0 | ml |

Preparation:

Active substance and mannitol are dissolved in water. After packaging the solution is freeze-dried. To produce the solution ready for use, the product is dissolved in water for injections.

### EXAMPLE 5

Dry Ampoule Containing 35 mg of Active Substance per 2 ml

Composition:

| | | |
|---|---|---|
| Active substance | 35.0 | mg |
| Mannitol | 100.0 | mg |
| water for injections | ad 2.0 | ml |

Preparation:

Active substance and mannitol are dissolved in water. After packaging, the solution is freeze-dried.

To produce the solution ready for use, the product is dissolved in water for injections.

### EXAMPLE 6

Tablet Containing 50 mg of Active Substance

Composition:

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

Preparation:

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 9 mm.

### EXAMPLE 7

Tablet Containing 350 mg of Active Substance

Composition:

| | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

(1), (2) and (3) are mixed together and granulated with an aqueous solution of (4). (5) is added to the dried granulated material. From this mixture tablets are pressed, biplanar, faceted on both sides and with a dividing notch on one side. Diameter of the tablets: 12 mm.

### EXAMPLE 8

Capsules Containing 50 mg of Active Substance

Composition:

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

Preparation:

(1) is triturated with (3). This trituration is added to the mixture of (2) and (4) with vigorous mixing.

This powder mixture is packed into size 3 hard gelatine capsules in a capsule filling machine.

### EXAMPLE 9

Capsules Containing 350 mg of Active Substance

Composition:

| | | |
|---|---|---|
| (1) Active substance | 350.0 | mg |
| (2) Dried maize starch | 46.0 | mg |
| (3) Powdered lactose | 30.0 | mg |
| (4) Magnesium stearate | 4.0 | mg |
| | 430.0 | mg |

Preparation:

(1) is triturated with (3). This trituration is added to the mixture of (2) and (4) with vigorous mixing.

This powder mixture is packed into size 0 hard gelatine capsules in a capsule filling machine.

### EXAMPLE 10

Suppositories Containing 100 mg of Active Substance

1 suppository contains:

| | | |
|---|---|---|
| Active substance | 100.0 | mg |
| Polyethyleneglycol (M.W. 1500) | 600.0 | mg |
| Polyethyleneglycol (M.W. 6000) | 460.0 | mg |
| Polyethylenesorbitan monostearate | 840.0 | mg |
| | 2000.0 | mg |

Preparation:

The polyethyleneglycol is melted together with polyethylene sorbitan monostearate. At 40° C. the ground active substance is homogeneously dispersed in the melt. It is cooled to 38° C. and poured into slightly chilled suppository moulds.

What is claimed is:

1. A compound of the formula I 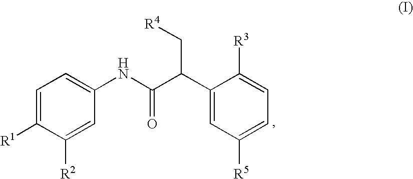
wherein:
R¹ denotes a C₃₋₇-cycloalkyl-carbonyl group, while
the methylene group in the 3 or 4 position in a C₅₋₇-cycloalkyl-carbonyl group may be replaced by a —NH group, wherein
the hydrogen atom of the —NH group may be replaced by a C₁₋₃-alkyl or C₁₋₃-alkylcarbonyl group,
a C₁₋₆-alkylcarbonyl group, optionally terminally substituted in the alkyl moiety by an amino, C₁₋₃-alkylamino or di-(C₁₋₃-alkyl)-amino group,
a group of formula R_{f}R_{g}N—(CH₂)ₘ—(Rₕ)N—CO, wherein
R_{f}, R_{g} and Rₕ independently of one another each denote a hydrogen atom or a C₁₋₃-alkyl group and
m denotes one of the numbers 2, 3 or 4,
a phenylcarbonyl, naphthylcarbonyl or heteroarylcarbonyl group,
while the phenyl, naphthyl or heteroaryl moiety may be substituted by a fluorine, chlorine or bromine atom, by a trifluoromethyl, C₁₋₃-alkyl, amino-C₁₋₃-alkyl, C₁₋₃-alkyl-amino-C₁₋₃alkyl, di-(C₁₋₃-alkyl)-amino-C₁₋₃-alkyl or C₁₋₃-alkoxy group,
a C₁₋₃-alkyl group substituted by a phenyl or heteroaryl group,
while the phenyl or heteroaryl substituent may be substituted by a fluorine, chlorine or bromine atom, by a trifluoromethyl, C₁₋₃-alkyl, amino-C₁₋₃-alkyl, C₁₋₃-alkyl-amino-C₁₋₃-alkyl, di-(C₁₋₃-alkyl)-amino-C₁₋₃-alkyl or C₁₋₃-alkoxy group,
a 2,5-dihydro-1H-pyrrol-1-ylcarbonyl group,
a 4- to 7-membered cycloalkyleneimino-carbonyl or cycloalkyleneimino-sulphonyl group optionally substituted by an amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group or
a group of formula 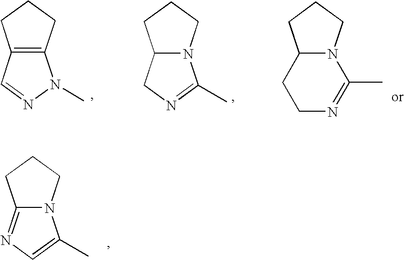
wherein in the heterocyclic moiety a hydrogen atom may be replaced by an aminomethyl or aminocarbonyl group in each case, p1 R² denotes a fluorine, chlorine or bromine atom, a C₂₋₃-alkenyl group or
a C₁₋₃-alkoxy or C₁₋₃-alkyl group wherein the hydrogen atoms may be wholly or partially replaced by fluorine atoms,
R³ denotes a hydroxy or amino group,
R⁴ denotes a phenyl or heteroaryl group which is optionally substituted by a hydroxy, C₁₋₄-alkyloxy, benzyloxy, hydroxycarbonyl-C₁₋₃-alkoxy, C₁₋₃-alkyloxy-carbonyl-C₁₋₃-alkyloxy, aminocarbonyl-C₁₋₃-alkyloxy, C₁₋₃-alkylaminocarbonyl-C₁₋₃-alkyloxy, di-(C₁₋₃-alkyl)-aminocarbonyl-C₁₋₃-alkyloxy, carboxy, C₁₋₃-alkyloxy-carbonyl group,
a 1-H-pyridonyl or 1-(C₁₋₃-alkyl)-pyridonyl group,
a 4- to 7-membered cycloalkyleneimino group or
a 4- to 7-membered cycloalkyl group wherein one or two methylene groups are replaced by a —NH or —N(C₁₋₃-alkyl)-group and wherein one or two of the methylene groups adjacent to the —NH or —N(C₁₋₃-alkyl)-group may each be replaced by a carbonyl group, with the proviso that a cycloalkyl group as hereinbefore defined wherein two —NH or —N(₁₋₃-alkyl)-groups are separated from one another by precisely one —CH₂-group is excluded, and
R⁵ denotes a group of formula —CH₂—NHR⁶, wherein
R⁶ denotes a hydrogen atom, a C₁₋₁₀-alkoxy-carbonyl, 2,2,2-trichloroethoxy-carbonyl, phenyloxycarbonyl or benzyloxycarbonyl group,
or a group of formula —C(═NH)—NH₂ wherein a hydrogen atom may be replaced by a C₁₋₁₀-alkoxy-carbonyl, 2,2,2-trichloroethoxycarbonyl, phenyloxycarbonyl, benzyloxycarbonyl, phenylcarbonyl, hydroxy, C₁₋₅-alkyloxy, benzyloxy or phenyloxy group,
while, unless otherwise stated, the term heteroaryl group denotes a monocyclic 5- or 6-membered heteroaryl group optionally substituted in the carbon skeleton by a C₁₋₃-alkyl, carboxy, C₁₋₃-alkoxy-carbonyl or C₁₋₃-alkoxy-carbonylamino group, while
the 6-membered heteroaryl group contains one, two or three nitrogen atoms and
the 5-membered heteroaryl group contains an imino group optionally substituted by a C₁₋₃-alkyl or phenyl-C₁₋₃-alkyl group, an oxygen or sulphur atom or
an imino group optionally substituted by a C₁₋₃-alkyl, amino-C₁₋₃-alkyl, C₁₋₃alkylamino-C₁₋₃-alkyl, di-(C₁₋₃-alkyl)-amino-C₁₋₃-alkyl or phenyl-C₁₋₃-alkyl group or an oxygen or sulphur atom and additionally contains a nitrogen atom or
an imino group optionally substituted by a C₁₋₃-alkyl or phenyl-C₁₋₃-alkyl group or an oxygen or sulphur atom and additionally contains two nitrogen atoms,
an imino group optionally substituted by a C₁₋₃-alkyl or phenyl-C₁₋₃-alkyl group and contains three nitrogen atoms,
and moreover a phenyl ring may be fused to the abovementioned monocyclic heterocyclic groups via two adjacent carbon atoms and the binding takes place via a nitrogen atom or via a carbon atom of the heterocyclic moiety or a fused-on phenyl ring,
while the abovementioned alkyl and alkoxy groups include straight-chain and branched alkyl and alkoxy groups, wherein additionally one to 3 hydrogen atoms may be replaced by fluorine atoms,
or a tautomer or pharmaceutically acceptable salt thereof.

2. A compound of the formula I according to claim 1, wherein:
R², R³, R⁴ and R⁵ are defined as in claim 1 and
R¹ denotes a 2,5-dihydro-1H-pyrrol-1-ylcarbonyl group,
a 4- to 7-membered cycloalkyleneimino-carbonyl group optionally substituted by an amino-C₁₋₃-alkyl, C₁₋₃-alkylamino-C₁₋₃-alkyl, di-(C₁₋₃-alkyl)-amino-C₁₋₃-alkyl, aminocarbonyl, C₁₋₃-alkylamino-carbonyl or di-(C₁₋₃-alkyl)-aminocarbonyl group or
a group of formula 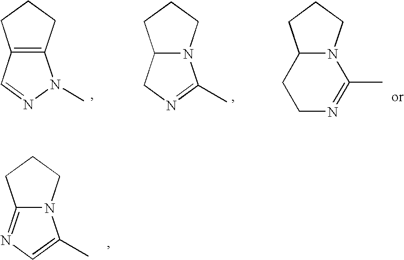
wherein in the heterocyclic moiety a hydrogen atom may be replaced by an aminomethyl or aminocarbonyl group in each case,
the abovementioned alkyl and alkoxy groups including straight-chain and branched alkyl and alkoxy groups, wherein additionally one to 3 hydrogen atoms may be replaced by fluorine atoms,
or a tautomer or pharmaceutically acceptable salt thereof.

3. A compound of the formula I in accordance with claim 2, wherein:
R¹, R², R³ and R⁵ are defined as in claim 2 and
R⁴ denotes a phenyl, furanyl, thienyl, pyrrolyl, pyrazolyl, pyridinyl, pyrazinyl, pyridazinyl, pyrimidinyl, thiazolyl or isoxazolyl group which is optionally substituted by a hydroxy, C₁₋₄-alkyloxy, benzyloxy, hydroxycarbonyl-C₁₋₃-alkoxy, C₁₋₃-alkyloxy-carbonyl-C₁₋₃-alkyloxy, aminocarbonyl-C₁₋₃-alkyloxy, C₁₋₃-alkylaminocarbonyl-C₁₋₃-alkyloxy, di-(C₁₋₃-alkyl)-aminocarbonyl-C₁₋₃-alkyloxy, carboxy, C₁₋₃-alkyloxy-carbonyl group,
the abovementioned alkyl and alkoxy groups including straight-chain and branched alkyl and alkoxy groups, wherein additionally one to 3 hydrogen atoms may be replaced by fluorine atoms,
or a tautomer or pharmaceutically acceptable salt thereof.

4. A compound selected from the group consisting of:
(a) 2-(5-amidino-2-hydroxy-phenyl)-N-[3-trifluoromethyl-4-(pyrrolidin-1-yl-carbonyl)-phenyl]-3-phenyl-propionamide
(b) 2-(5-amidino-2-hydroxy-phenyl)-N-[3-trifluoromethyl-4-(pyrrolidin-1-yl-carbonyl)-phenyl]-3-(pyridin-3-yl)-propionamide, and
(c) 2-(5-aminomethyl-2-hydroxy-phenyl)-N-[3-trifluoromethyl-4-(pyrrolidin-1-yl-carbonyl)-phenyl]-3-phenyl-propionamide,
or an analog of compound (a), (b) or (c) wherein the amidino group is substituted by a hydroxy, C₁₋₅-alkyloxy, C₁₋₁₀-alkoxy-carbonyl or phenylcarbonyl group,
or a pharmaceutically acceptable salt thereof.

5. A pharmaceutical composition comprising a compound in accordance with claim 1, 2, 3 or 4 and one or more inert carriers and/or diluents.

6. A method for treating or inhibiting thrombus formation which comprises administering an antithrombotic amount of a compound in accordance with claim 1, 2, 3, or 4.
